(19) United States
(12) Patent Application Publication
(10) Pub. No.: US 2017/0021074 A1
(43) Pub. Date: Jan. 26, 2017

(54) Implantable pump system, as well as a mehod for bringing a pump system to a location application

(71) Applicants: Berlin Heart GmbH, Berlin (DE); Universität Zürich, Zurich (CH)

(72) Inventors: Ulrich Tim Opfermann, Berlin (DE); Andreas Arndt, Berlin (DE); Volkmar Falk, Berlin (DE)

(21) Appl. No.: 15/215,076

(22) Filed: Jul. 20, 2016

(30) Foreign Application Priority Data
Jul. 20, 2015 (EP) 15177575.6

(51) Int. Cl. A61M 1/12; A61M 1/10

(52) U.S. Cl. CPC A61M 1/125; A61M 1/122; A61M 1/1096; A61M 1/1031

(57) ABSTRACT

The invention relates to an implantable pump system for delivering blood within the body of a patient, with a blood pump which delivers a fluid in an axial direction and comprises a rotatingly drivable rotor as well as a pump casing surrounding the rotor, as well as a support tube, in which the pump casing is arranged and held, wherein an annular gap is formed between the support tube and the pump casing. An almost physiological blood flow is rendered possible in this manner, by way of the combination of a flow through the pump casing on the one hand, and the annular gap on the other hand.

The present invention lies in the field of mechanics and is applicable particularly advantageously in medical technology in the field of implanting pumps.

The invention is particularly advantageously applicable in the field of blood pumps assisting the delivery of blood in the circulation system of a living being within blood vessels, and, as the case may be, between a heart and adjacent blood vessels.

Implantable blood pumps for assisting the left heart ventricle (left ventricular assist devices, LVAD) are known which are connected within the pericardium (interpericardial placement) from the outside onto the left ventricle by way of a cannula and even completely or partly project into the left ventricle (intraventricular or partial intraventricular placement). The pump takes blood from the left ventricle and usually delivers it via a relatively long outlet cannula into the aorta. These pumps have the advantage of an adequate delivery output with a relatively low degree of damage to the blood. The invasive implantation manner which demands a median stemotomy or at least a thoractomy is however disadvantageous. The relatively large blood contact surface, combined with an excitation of the coagulation system, is likewise disadvantageous. Intraventricular or partially intraventricular pumps, apart from a relatively large contact surface with the blood, moreover have the problem that a rigid anchoring of the pump in the apex can induce injury, inflammation reactions and tissue proliferation in the case of relative movements of the pump and of the cannula to the endocardium, to the aortic valve and to the inner wall of the aorta. Here as well, thromboembolic complications may be the consequence.

Against the background of the state of the art, it may be an object of the present invention to create a stable and reliable implantable pump system, with which the surface coming into contact with the fluid to be delivered can be kept small, and with which, when used as a heart assistance pump, a blood flow that is as physiologically compatible as possible can be produced or is rendered possible.

The object may be achieved by the features of the invention according to the patent claims.

The invention thus specifically relates to an implantable pump system for delivering blood within a body of a patient having a blood pump which delivers a fluid in an axial direction and which comprises a rotatingly driveable rotor as well as a pump casing surrounding the rotor.

According to the present invention, it is provided that the pump system additionally comprises a support tube in which the pump casing is arranged and held, wherein an annular gap is formed between the support tube and the pump casing.

On account of the design according to an embodiment of the invention, on the one hand fluid, in particular blood, can be delivered in the axial direction in the inside of the pump casing, in particular by a rotor. This delivery of blood can be accomplished largely independently of the physiologically built-up blood pressure, thus for example independently of the phase of the heart rhythm.

According to the design according to an embodiment of the invention, a further flow which is independent of the direct delivery through the blood pump is however additionally possible through the annular gap between the pump casing and the support tube. The annular gap can be at least 0.3 mm, in particular at least 0.5 mm, advantageously 1.5 to 3.5 mm wide, in particular in the radial direction. This blood flow through the annular gap can correspond for example to the blood flow delivered by the heart activity. On account of this, the activity of the heart is not compromised, which is to say it is independent of the assistance by machine, and a physiologically naturally controlled blood flow is superimposed on the blood flow produced by the blood pump. This can have very useful effects, in particular with respect to the pulsation of the blood flow, since the emergence of a stationary flow is prevented, and corresponding non-stationary flow conditions are produced and deadwater regions of the blood flow are therefore avoided. The function of the blood pressure control mechanisms in the patient's body is also largely held intact by way of this. A part of the volume flow delivered by the heart can use this annular gap as a bypass to the pump in the case of a high delivery output of the heart. This moreover provides the advantage that this flow path continues to be available in the case of any failure of the pump.

The annular gap between the support tube and the pump casing can be shaped as an annulus, but the blood pump can also be displaced in the radial direction with respect to the middle axis of the support tube, so that an irregularly shaped annular gap arises.

The blood pump itself can be desired in a rigid manner, i.e. in a manner in which it is not radially compressible, or can be designed in a radially compressible manner. In the case of a non-compressible blood pump, the support tube, for implantation, is compressed radially to such an extent that it surrounds the pump casing of the blood pump as snugly as possible. If the blood pump itself is compressible, then the support tube can be radially compressed to a further extent, so that the support tube as well as the casing of the blood pump and, as the case may be, also the rotor of the blood pump are radially compressed on impanation. With the implantation procedure, the support tube after being pushed out of the hollow catheter will then expand radially to a greater extent than the pump casing of the blood pump, so that a corresponding annular gap arises between the support tube and the pump casing.

The annular gap can comprise a valve device which permits a fluid to pass the annular gap in the axial direction in a first flow direction and blocks the annular gaps with respect to the opposite second flow direction, so as to prevent the blood which the pump and/or the heart deliver from flowing back through the annular gap. By way of such a valve device, it is ensured that on placing the pump system in the region of the aortic valve, blood can flow in only one direction, and specifically out of the left ventricle, but not back into this via the annular gap. The valve device closes a flow direction in the region of the annular gap for this reason. The valve device can be designed as a check flap valve.

However, it is also advantageously conceivable for a second valve device to release or to block the cross section of the pump casing. In this case, the valve device exclusively controls the physiological flow within the blood pump, even when the blood pump does not maintain the pressure gradient between the ventricle and the aorta, which can be the case with any sort of defect of the pump or in the case of too low a delivery power of the pump, and thus prevails the backflow of blood into the ventricle through the pump casing.

It is also conceivable to make do without any valve device, and to prevent a backflow or a significant backflow through the annular gap by way of an advantageous design of the flow characteristics in the region downstream of the pump.

The support tube, as described above, can basically be expandable in the radial direction. This can simplify the placement with different implantation methods.

The pump together with the support tube is envisaged for implantation in the region of the aortic valve and should advantageously be advanced through the apex of the left ventricle up to into the region of the aortic valve and be anchored there.

One advantageous embodiment of the support tube envisages this being designed as a stent, in order to permit this anchoring. This stent can advantageously be provided with holding anchors which can ensure an axial positioning and anchoring of the support tube in the aortic valve sinus on implantation, whereas the support wires of the stent, which are basically released after the holding anchors on advancing the stent out of the hollow catheter, effect a pressing of the aortic valve cusps against the aorta wall and a clamping of the support tube in the aorta.

The holding anchors and support wires are usually designed as spring-elastic wires, in particularly of a shape memory alloy material such as nitinol.

The support tube can moreover be advantageously designed as a foldable wire mesh or can comprise such a foldable wire mesh. The support tube is simply and reversibly radially compressible and expandable by way of such a design. Such a wire mesh can also be simply fixed on the aorta wall by way of elastic clamping.

According to an embodiment of the invention, the blood pump can advantageously be designed in a manner such that it comprises a rotor which delivers blood in the axial direction and which is rotatably mounted in the pump casing in at least one bearing, in particular in two bearings. Corresponding rotation bearings which are advantageously designed as plane bearings (hydrodynamic bearings), contact bearings, magnetic bearings, or a combination of these, can be provided for example at an inflow opening and/or onflow opening of the pump casing.

Such a rotor of a blood pump can be driven for example by way of a drivable, flexible shaft which runs through a hollow catheter, for example within the aorta or another blood vessel, up to a lock and from there up to a motor which can be arranged within or outside the patient's body.

However, one can also advantageously envisage the blood pump comprising a motor in the region of the support tube, in particular within the support tube. The motor can be provided directly on the rotor for this. Such a motor can be designed for example as an electric motor or also as a turbine which is pneumatically or hydraulically drivable by way of a fluid flow.

Accordingly, according to an embodiment of the invention, one can also envisage the motor being connected to an energy source by way of a lead/conduit. Such a lead/conduit according to the above embodiments can either be an electrical lead, or a fluid conduit which supplies the motor arranged in the support tube with a fluid or gas flow for driving the turbine.

Such a lead/conduit can extend from the motor out of the support tube in the axial direction either along a blood vessel/the aorta, up to a suitable lock and from there to an energy source which can be arranged within or outside the body of the patient. The lead/conduit can also however extend from the motor out of the support tube into a ventricle and from there transapically through a feed-through out of the heart to an energy source within or outside the patient's body.

Both possibilities of leading the conduit/lead are physiologically possible and provide different advantages depending on the methods of the implantation of the pump system.

The invention furthermore, apart from a pump system of the type described above, also relates to a hollow catheter with a radially compressed support tube which is arranged in this hollow catheter, as well as with a blood pump which is fixed in the support tube and comprises a rotor and a pump casing. Such a hollow catheter can be industrially premanufactured and pre-prepared and contain a radially compressed support tube with a blood pump which is located in this and Which is pre-sterilised and prepared for implantation. The blood pump can either likewise be radially compressed or also non-compressed within the support tube.

A corresponding hollow catheter can also be prepared shortly before an implantation, by way of drawing in a compressed support tube together with a blood pump which is suitably contained therein.

A hollow catheter for the applications mentioned above for example has an outer diameter which permits it to be pushed through an aortic valve with a gap, so that the physiological flow in the region between the hollow catheter and the aortic valve is not compromised too much during the implantation procedure.

The invention moreover relates to a port system for implanting a blood pump system into the apex of the left ventricle of the heart, with a first catheter feed-through as well as a hollow catheter which is axially displaceable through the feed-through. The hollow catheter itself corresponds to the design mentioned above.

First, the feed-through is applied in a ventricle wall lying roughly opposite the aortic valve (i.e. in the apex), in a minimal-invasive operation, so that the hollow catheter can be pushed through the feed-through into the ventricle to the aortic valve and pushed into this. The support tube is applied there and the hollow catheter is slowly retracted. The support tube on sliding out of the hollow catheter first releases holding anchors which anchor the support tube within the aortic valve sinus in the axial direction. The support wires effecting a radial fixation by way of clamping on the aorta wall are subsequently released, pressing the aortic valve cusps against the aorta wall.

The hollow catheter can then be retracted through the feed-through, the energy supply conduit/lead leading through the ventricle and though the feed-through out of the heart to an energy source being released by the retracted hollow catheter. The feed-through in the heart wall can then be closed, for example by way of a membrane with an opening receiving the energy lead/conduit and in particular a sleeve seal, in a manner such that this feed-through is sealed around the led-through energy lead/conduit.

Accordingly, an advantageous method for bringing a pump system to an application location envisages a hollow catheter with a radially compressed support tube as well as a pump, arranged therein and having a rotor and a pump casing, first being led through a feed-through, whereupon the support tube is pushed out of the hollow catheter and expanded in a manner such that an annular gap arises between the support tube and the pump casing. The subject-matter of the present patent application, apart from the mentioned method, also includes a device for carrying out the method or also individual elements of such a device, which serve for carrying out a method described above.

In conclusion, it can be said that the present patent application relates to an implantable pump system, in particular a transapically implantable pump system, as well as to a hollow catheter and a port system, for implantation of a blood pump system described above. A suitable method for bringing a pump system to a location of application is also referred to. In conclusion, it is to be noted that the mentioned subject-matter can relate to applications in the human or animal body. With this, basically a new type of VAD (ventricular assist device) is put forward These can either be applied as an LVAD (left ventricular assist device) or as an RVAD (right ventricular assist device). The pump system is also envisaged for complete intravasal implantation. An almost physiological blood flow is rendered possible in this manner by way of the combination on the one hand of a flow through the pump casing according to an embodiment of the invention and on the other hand the annular gap in the pump system, by which means it is possible to do without extravasal blood paths.

The invention is hereinafter represented by way of an exemplary embodiment in several figures, and is explained hereinafter. There is shown in

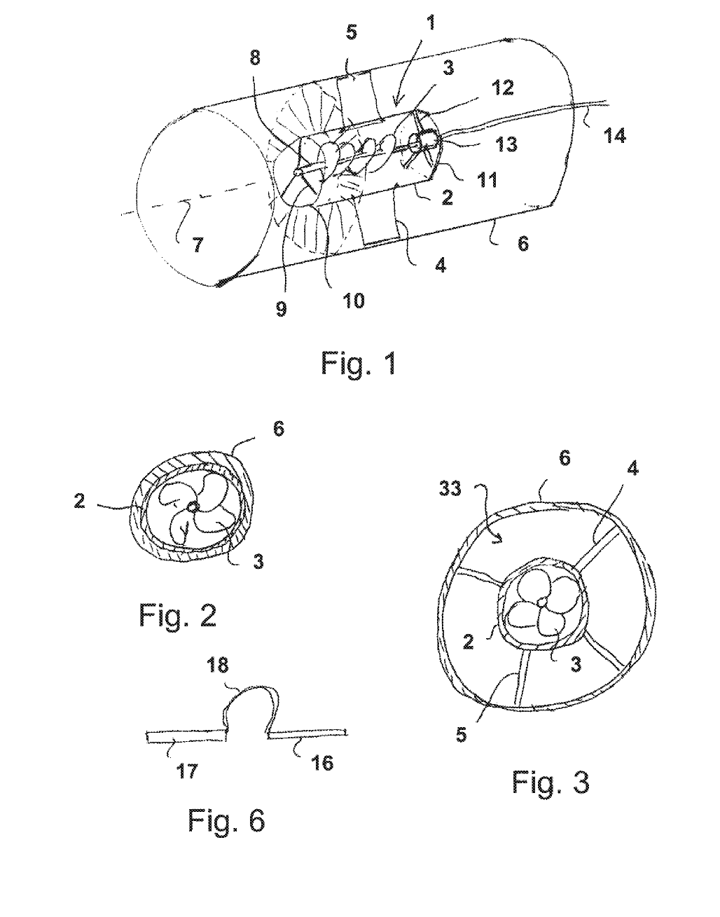

FIG. 1 schematically, a support tube with a pump arranged therein, in a three-dimensional view,

FIG. 2 a cross section through a radially compressed support pump with a pump arranged therein,

FIG. 3 a cross section of a radially expanded support tube, with a pump arranged therein,

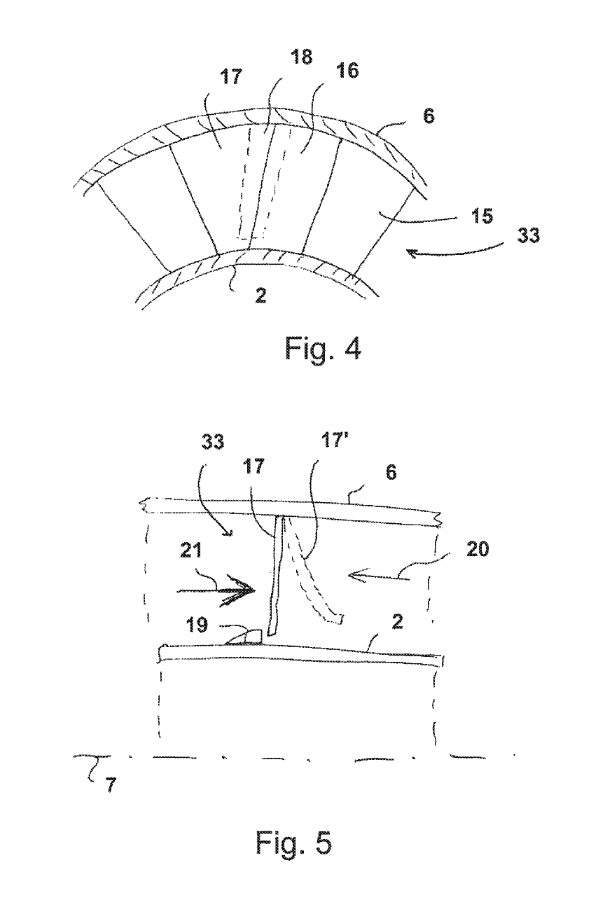

FIG. 4 a plan view of the annular gap between a support tube and a pump, in a segmented detail,

FIG. 5 a longitudinal section through a support tube with a part of a pump and a valve device,

FIG. 6 a view of a part of a valve device in the radial direction with respect to the support tube,

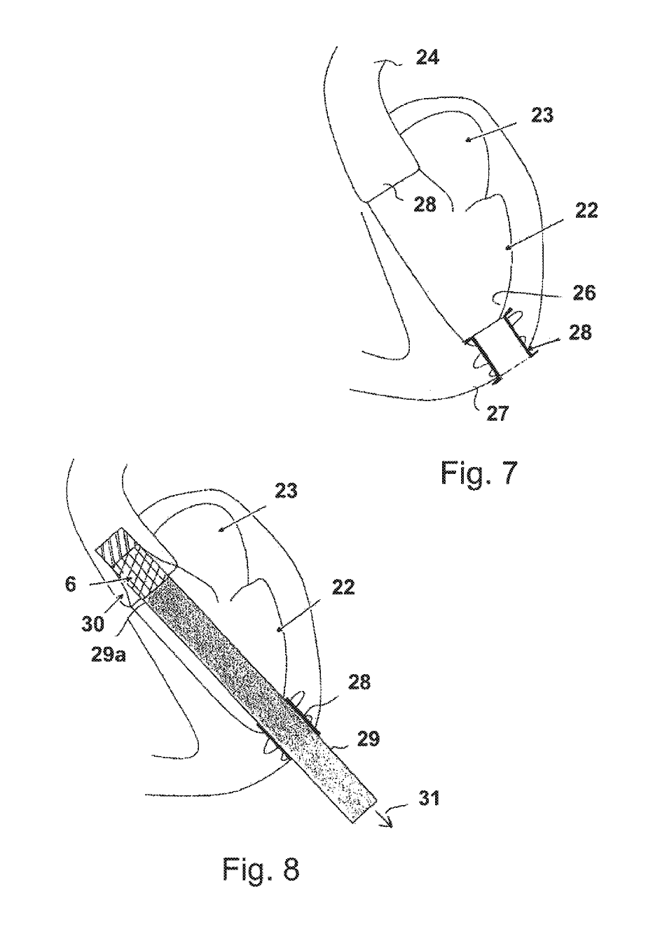

FIG. 7 the arrangement of a port system in a human heart,

FIG. 8 a port system with a hollow catheter and with a pump system which is placed in the region of the aortic valve of a heart,

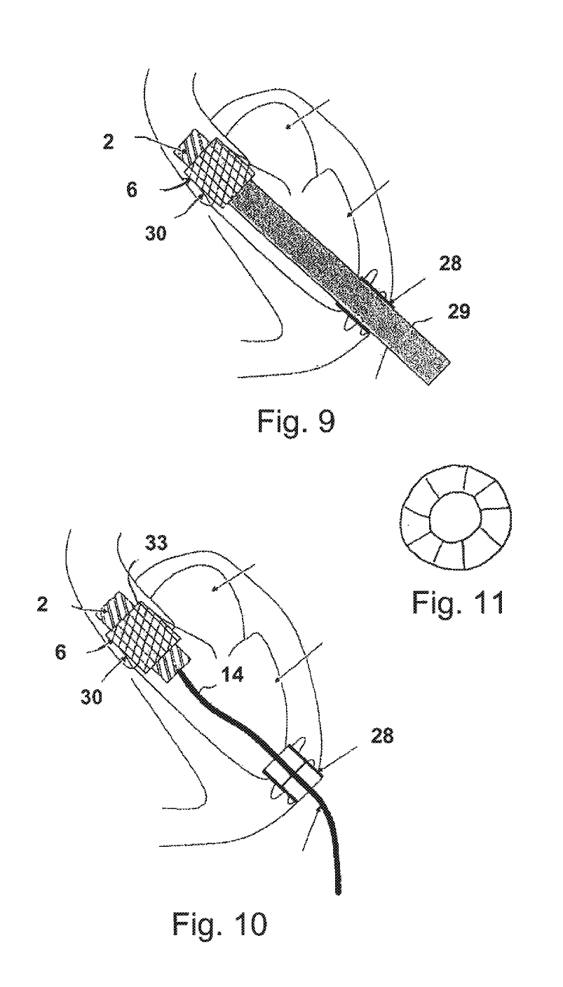

FIG. 9 the pump system of FIG. 8, with an expanded support tube,

FIG. 10 the pump system of FIG. 9, after retracting the hollow catheter as well as

FIG. 11 a plan view upon the support tube, as well as a valve device.

FIG. 1 in a three-dimensional view shows a blood pump 1 which comprises a pump casing 2 as well as a rotor 3, and is held in a support tube 6 in the form of a stent, by way of webs 4, 5.

The pump casing 2 is constructed in a cylindrical or rotationally symmetrical manner with respect to an axis 7 which also coincides with the middle axis of the shaft 8 of the rotor 3. The middle axis 7 moreover indicates the axial direction of the arrangement. The support tube 6 just as the pump casing 2 is constructed in a rotationally symmetrical manner and concentrically surrounds this.

The shaft 8 of the rotor 3 is fixed in the pump casing 2 by way of webs 9 within the pump casing 2. The webs 9 are arranged at a first end 10 of the pump casing 2. Further webs 12 which centrally fix a motor 13 designed as an electric motor in the pump casing are arranged at the second end 11 of the pump casing 2. The shaft of the motor 13 is connected to or is identical to the shaft 8 of the rotor 3. Alternative means for mounting and driving the rotor as well as the arrangement of inlet or outlet guide vanes are likewise included by the the subject-matter of the present property right.

The motor 13 is supplied with electrical energy by way of a lead 14 and drives the rotor 3. The rotor 3 comprises one or more delivery elements in the form of blades which deliver a fluid in the axial direction on rotation about the middle axis 7.

The rotor 3 as well as the pump casing can be radially compressible, so that they assume a smaller space on transport to the place of application, than on operation. The pump casing and the rotor however can also be designed in a rigid manner. The support tube 6 can likewise be constructed in a radially compressible manner, in particular if it is designed in the manner of a stent as a foldable wire mesh. The support tube 6 in this case can be radially compressed to such an extent that it snugly surrounds the pump casing 2, for transport.

This condition is shown in more detail in a cross-sectional representation in FIG. 2. There, the support tube 6 is represented in a radially compressed form, surrounding the pump casing 2 in a direct manner. The pump casing 2 is not radially compressed and surrounds the rotor 3 which is likewise not radially compressed.

In this condition, the pump system can be brought to the location of application in a simple manner and with little operative effort with regard to the patient. For this, it can firstly be brought into a hollow catheter, as will be explained in more detail further below, and then displaced with the hollow catheter.

FIG. 3 in cross section shows a pump system with a non-compressed support tube 6 which concentrically surrounds a pump casing 2 with a rotor 3 whilst forming an annular gap 33. Moreover, webs 4, 5 are represented, and these are foldable in a manner such that they do not prevent the radial compression of the support tube 6. The webs 4, 5 for example can consist of a spring elastic or of a limp material so that they can only be loaded in tension on fixing the pump casing 2 in the support tube 6.

A segment of the support tube 6 is represented in cross section in FIG. 4, together with a segment of the pump casing 2. A valve device comprising several flap segments 15, 16, 17 is arranged in the annular gap 33, between the support tube and the pump casing, wherein the flap segments are fastened in a hinge-like manner on one of the two parts, thus either on the support tube 6 or on the pump casing 2, and can pivot out in the axial direction, in order to release the annular gap for a fluid flow.

If the flap segments are aligned perpendicularly to the middle axis 7, they are then in tight contact with one another and completely block the annular gap.

Flap pockets, of which one is represented in FIG. 6 by way of example, can be provided between them. There, considered in the radial direction, two flap segments 17, 16 with a flap pocket 18 are represented, and this flap pocket flexibly connects the two flap segments 16, 17 to one another at least over a part of their length, in particular however also over the whole length, in the manner of a film hinge. The flap pocket 18 is represented in FIG. 4 in a dashed manner.

It is represented in FIG. 5 that in the idle position, represented by unbroken lines, the segments 17 are aligned perpendicularly to the middle axis 7 between the support tube 6 and the pump casing 2. In this position, the flap segments 17 abut an annular abutment 19 which is fastened to the pump casing 2 concentrically at the outside. Through this, the segments 17 create a resistance counter to a flow in the direction of the arrow 20. A flow is let through in the opposite direction, indicated by the arrow 21, by way of it moving the segments 17 into the deflected-out position 17′, represented in a dashed manner, and thus opening the annular gap 33.

A check valve is therefore realised for the region of the annular gap 33, and this check valve permits a fluid flow, in particular a blood flow in only one flow direction, and blocks it in the opposite direction, wherein the flow in the centric part of the pump system, between the ends 10, 11 of the pump casing 2, is determined exclusively by the drive by way of the pump rotor. However, it is also conceivable to also control the flow in the region of the pump casing by way of a separate check valve.

A different embodiment of the check valve in the form of a cusp valve, as is known for aortic valve replacement, is also conceivable. Also several cusps can be applied instead of the known three cusps.

FIG. 7, as a typical application location for the pump system according to an embodiment of the invention, shows a human heart, and specifically more precisely the left ventricle 22, the left atrium 23, the ascending aorta 24 and the aortic valve region 25.

A feed-through 28 is inserted into the heart wall 17, in the region of the apex 26, opposite the aortic valve region 25. The feed-through for example can be designed as a pump branch (stub) with two flanges projecting radially outwards on both sides of the heart wall 27. The feed-through comprises a closure mechanism, so that after use, it can be closed for restoring the functioning capability of the left ventricle.

A left heart assist system in the form of the pump system according to an embodiment of the invention is to be placed in the region of the aortic valve 25.

In FIG. 8, it is shown that a hollow catheter 29 is inserted from the side of the heart which is opposite the aorta, through the feed-through 28 into the left ventricle 22 and is pushed through this, so that the distal end 29a of the hollow catheter with the compressed support tube and the blood pump is placed in the region of the aortic valve. The holding anchors 30 in this position can detach from the support tube 6 with the stepwise retraction of the hollow catheter, and anchor in the region of the aortic sinus.

Parts of the cusps of the aortic valve are pressed onto the aorta wall if the hollow catheter 29 is retracted further in the direction of the arrow 31. If the catheter completely releases the support tube 6 in FIG. 9, then this support tube can expand further radially and press the valve cusps completely onto the aorta wall and seize there.

Through this, the annular gap 33 between the support tube 6 and the pump casing 2 as described above arises. In the example shown, the pump casing 2 projects axially beyond the support tube. The pump casing can project axially beyond the support tube 6 at one side, or at both sides as in the example shown and as evident in FIG. 10. The support tube, however, can also be designed longer that the pump casing.

The length of the pump casing 2 in the case described here is significantly larger than the length of the support tube 6. If the hollow catheter 29 is retracted even further with respect to the position represented in FIG. 9 and is removed through the feed-through 29, then the energy lead/conduit 14 led from the motor of the pump rotor through the left ventricle and the feed-through 28 out of the heart and connected to an energy source remains, which energy source is either implanted within the patient's body or is positioned outside the patient's body. The feed-through 28 can be closed to such an extent that it seals the heart around the conduit 14.

Blood can be subsequently ejected through the annular gap 33 between the pump casing 2 and the support tube 6 and be transported into the aorta, according to the physiological function of the heart, wherein the check valve seals off the annular imp in the low pressure phase of the heart, as described above. The central pump which is arranged in support tube 6 moreover constantly delivers blood out of the left ventricle into the aorta by way of the rotating rotor. The output of the pump can be modulated in a rhythmic manner in accordance with the pulsating delivery output of the heart, in order to produce a physiologically normal flow pattern, or the output of this pump can also be kept constant.

With the described pump system, cannulae are neither necessary in the suction region nor in the ejection (delivery) region, so that the total surface which is wetted by the blood can be kept low. An almost physiological, pulsating formation of the blood flow in the aorta is moreover possible.

The incorporation of the pump system into the heart of a patient can be effected transapically with little operative effort, or also through a blood vessel. Perioperative risks are thus likewise minimised.

1. A transapically implantable pump system for delivering blood within a body of a patient, with a blood pump which delivers a fluid in an axial direction and comprises a rotatingly drivable rotor as well as a pump casing surrounding the rotor, wherein the pump casing is arranged and held in a support tube, wherein an annular gap is formed between the support tube and the pump casing.

2. The pump system according to claim 1, wherein a valve device which is arranged in the annular gap and which lets a fluid pass the annular gap in the axial direction in a first flow direction, and blocks the annular gap with respect to the opposite, second flow direction.

3. The pump system according to claim 1, wherein the support tube is configured such that it presses the cusps of the aortic valve into the aorta wall and fixes itself at this position during implantation into a body of a patient.

4. The pump system according to claim 1, wherein the support tube is expandable in the radial direction.

5. The pump system according to claim 1, wherein the support tube is configured as a stent.

6. The pump system according to claim 1, wherein the support tube is provided with holding anchors for the axial positioning.

7. The pump system according to claim 1, wherein the support tube is provided with support wires for the radial anchoring.

8. The pump system according to claim 1, wherein the support tube comprises a foldable wire mesh.

9. The pump system according to claim 1, wherein the blood pump comprises a rotor which delivers blood in the axial direction and which is rotatably mounted in the pump casing, in at least one bearing, in particular in two bearings.

10. The pump system according to claim 1, wherein the blood pump comprises a motor in the region of the support tube, in particular within the support tube.

11. The pump system according to claim 10, wherein the motor is connected to an energy source by way of a lead/conduit.

12. The pump system according to claim 11, wherein the lead/conduit extends out of the support tube in the axial direction.

13. A hollow catheter with a radially compressed support tube which is arranged therein, as well as with a blood pump which is fixed in the support tube and which comprises a rotor and a pump casing.

14. The hollow catheter of claim 13, included in a port system for implanting a blood pump system into the apex of the left ventricle of the heart, with a first catheter feed-through as well as with the hollow catheter, said hollow catheter being axially displaceable through the feed-through.

15. The hollow catheter of claim 14, included in the port system, wherein the feed-through comprises a valve which is designed such that it seals the port against blood running out, before the advance and after the retraction of the hollow catheter.

16. A method for placing a pump system at a location of application, in which first a hollow catheter with a radially compressible support tube as well as with a pump which is arranged in this and which has a rotor and a pump casing is led through a feed-through, the support tube is subsequently displaced out of the hollow catheter and radially expanded in a manner such that an annular gap arises between the support tube and the pump casing.

17. The pump system according to claim 1, wherein the pump together with support tube is designed such that said support tube is implantable in the region of the aortic valve and replaces the aortic valve.
